SEQUENCE LISTING (28 sequences)

SEQ ID NO: 1 - DNA, 3446 bp, Homo sapiens
  source 1..3446: mol_type = genomic DNA
gtctcagttcccgagcctgggagcaaccgcagcttctagtatccagactccagcgccgccccgggcgcggaccccaaccccgacccagagcttctccagcggcggcgcagcgagcagggctccccgccttaacttcctccgcggggcccagccaccttcgggagtccgggttgcccacctgcaaactctccgccttctgcacctgccacccctgagccagcgcgggcgcccgagcgagtcatggccaacgcggggctgcagctgttgggcttcattctcgccttcctgggatggatcggcgccatcgtcagcactgccctgccccagtggaggatttactcctatgccggcgacaacatcgtgaccgcccaggccatgtacgaggggctgtggatgtcctgcgtgtcgcagagcaccgggcagatccagtgcaaagtctttgactccttgctgaatctgagcagcacattgcaagcaacccgtgccttgatggtggttggcatcctcctgggagtgatagcaatctttgtggccaccgttggcatgaagtgtatgaagtgcttggaagacgatgaggtgcagaagatgaggatggctgtcattgggggtgcgatatttcttcttgcaggtctggctattttagttgccacagcatggtatggcaatagaatcgttcaagaattctatgaccctatgaccccagtcaatgccaggtacgaatttggtcaggctctcttcactggctgggctgctgcttctctctgccttctgggaggtgccctactttgctgttcctgtccccgaaaaacaacctcttacccaacaccaaggccctatccaaaacctgcaccttccagcgggaaagactacgtgtgacacagaggcaaaaggagaaaatcatgttgaaacaaaccgaaaatggacattgagatactatcattaacattaggaccttagaattttgggtattgtaatctgaagtatggtattacaaaacaaacaaacaaacaaaaaacccatgtgttaaaatactcagtgctaaacatggcttaatcttattttatcttctttcctcaatataggagggaagatttttccatttgtattactgcttcccattgagtaatcatactcaactgggggaaggggtgctccttaaatatatatagatatgtatatatacatgtttttctattaaaaatagacagtaaaatactattctcattatgttgatactagcatacttaaaatatctctaaaataggtaaatgtatttaattccatattgatgaagatgtttattggtatattttctttttcgtctatatatacatatgtaacagtcaaatatcatttactcttcttcattagctttgggtgcctttgccacaagacctagcctaatttaccaaggatgaattctttcaattcttcatgcgtgcccttttcatatacttattttattttttaccataatcttatagcacttgcatcgttattaagcccttatttgttttgtgtttcattggtctctatctcctgaatctaacacatttcatagcctacattttagtttctaaagccaagaagaatttattacaaatcagaactttggaggcaaatctttctgcatgaccaaagtgataaattcctgttgaccttcccacacaatccctgtactctgacccatagcactcttgtttgctttgaaaatatttgtccaattgagtagctgcatgctgttcccccaggtgttgtaacacaactttattgattgaatttttaagctacttattcatagttttatatccccctaaactacctttttgttccccattccttaattgtattgttttcccaagtgtaattatcatgcgttttatatcttcctaataaggtgtggtctgtttgtctgaacaaagtgctagactttctggagtgataatctggtgacaaatattctctctgtagctgtaagcaagtcacttaatctttctacctcttttttctatctgccaaattgagataatgatacttaaccagttagaagaggtagtgtgaatattaattagtttatattactctcattctttgaacatgaactatgcctatgtagtgtctttatttgctcagctggctgagacactgaagaagtcactgaacaaaacctacacacgtaccttcatgtgattcactgccttcctctctctaccagtctatttccactgaacaaaacctacacacataccttcatgtggttcagtgccttcctctctctaccagtctatttccactgaacaaaacctacgcacataccttcatgtggctcagtgccttcctctctctaccagtctatttccattctttcagctgtgtctgacatgtttgtgctctgttccattttaacaactgctcttacttttccagtctgtacagaatgctatttcacttgagcaagatgatgtaatggaaagggtgttggcattggtgtctggagacctggatttgagtcttggtgctatcaatcaccgtctgtgtttgagcaaggcatttggctgctgtaagcttattgcttcatctgtaagcggtggtttgtaattcctgatcttcccacctcacagtgatgttgtggggatccagtgagatagaatacatgtaagtgtggttttgtaatttaaaaagtgctatactaagggaaagaattgaggaattaactgcatacgttttggtgttgcttttcaaatgtttgaaaacaaaaaaaatgttaagaaatgggtttcttgccttaaccagtctctcaagtgatgagacagtgaagtaaaattgagtgcactaaacaaataagattctgaggaagtcttatcttctgcagtgagtatggcccgatgctttctgtggctaaacagatgtaatgggaagaaataaaagcctacgtgttggtaaatccaacagcaagggagatttttgaatcataataactcataaggtgctatctgttcagtgatgccctcagagctcttgctgttagctggcagctgacgctgctaggatagttagtttggaaatggtacttcataataaactacacaaggaaagtcagccactgtgtcttatgaggaattggacctaataaattttagtgtgccttccaaacctgagaatatatgcttttggaagttaaaatttaaatggcttttgccacatacatagatcttcatgatgtgtgagtgtaattccatgtggatatcagttaccaaacattacaaaaaaattttatggcccaaaatgaccaacgaaattgttacaatagaatttatccaattttgatctttttatattcttctaccacacctggaaacagaccaatagacattttggggttttataataggaatttgtataaagcattactctttttcaataaattgttttttaatttaaaaaaa

SEQ ID NO: 2 - protein, 211 aa, Homo sapiens
MANAGLQLLGFILAFLGWIGAIVSTALPQWRIYSYAGDNIVTAQAMYEGLWMSCVSQSTGQIQCKVFDSLLNLSSTLQATRALMVVGILLGVIAIFVATVGMKCMKCLEDDEVQKMRMAVIGGAIFLLAGLAILVATAWYGNRIVQEFYDPMTPVNARYEFGQALFTGWAAASLCLLGGALLCCSCPRKTTSYPTPRPYPKPAPSSGKDYV

SEQ ID NO: 3 - protein, 15 aa, Homo sapiens
QEFYDPLTPINARYE

SEQ ID NO: 4 - protein, 28 aa, Homo sapiens
SCVSQSTGQIQCKVFDSLLNLNSTLQAT

SEQ ID NO: 5 - protein, 18 aa, Homo sapiens
GSQIYMICNQFYTPGGTG

SEQ ID NO: 6 - protein, 27 aa, synthetic construct
SSVSQSTGQIQSKVDSLLNLNSTQATR

SEQ ID NO: 7 - protein, 230 aa, Homo sapiens
MASLGLQLVGYILGLLGLLGTLVAMLLPSWKTSSYVGASIVTAVGFSKGLWMECATHSTGITQCDIYSTLLGLPADIQAAQAMMVTSSAISSLACIISVVGMRCTVFCQESRAKDRVAVAGGVFFILGGLLGFIPVAWNLHGILRDFYSPLVPDSMKFEIGEALYLGIISSLFSLIAGIILCFSCSSQRNRSNYYDAYQAQPLATRSSPRPGQPPKVKSEFNSYSLTGYV

SEQ ID NO: 8 - protein, 220 aa, Homo sapiens
MSMGLEITGTALAVLGWLGTIVCCALPMWRVSAFIGSNIITSQNIWEGLWMNCVVQSTGQMQCKVYDSLLALPQDLQAARALIVVAILLAAFGLLVALVGAQCTNCVQDDTAKAKITIVAGVLFLLAALLTLVPVSWSANTIIRDFYNPVVPEAQKREMGAGLYVGWAAAALQLLGGALLCCSCPPREKKYTATKVVYSAPRSTGPGASLGTGYDRKDYV

SEQ ID NO: 9 - protein, 209 aa, Homo sapiens
MASMGLQVMGIALAVLGWLAVMLCCALPMWRVTAFIGSNIVTSQTIWEGLWMNCVVQSTGQMQCKVYDSLLALPQDLQAARALVIISIIVAALGVLLSVVGGKCTNCLEDESAKAKTMIVAGVVFLLAGLMVIVPVSWTAHNIIQDFYNPLVASGQKREMGASLYVGWAASGLLLLGGGLLCCNCPPRTDKPYSAKYSAARSAAASNYV

SEQ ID NO: 10 - protein, 218 aa, Homo sapiens
MGSAALEILGLVLCLVGWGGLILACGLPMWQVTAFLDHNIVTAQTTWKGLWMSCVVQSTGHMQCKVYDSVLALSTEVQAARALTVSAVLLAFVALFVTLAGAQCTTCVAPGPAKARVALTGGVLYLFCGLLALVPLCWFANIVVREFYDPSVPVSQKYELGAALYIGWAATALLMVGGCLLCCGAWVCTGRPDLSFPVKYSAPRRPTATGDYDKKNYV

SEQ ID NO: 11 - protein, 220 aa, Homo sapiens
MASAGMQILGVVLTLLGWVNGLVSCALPMWKVTAFIGNSIVVAQVVWEGLWMSCVVQSTGQMQCKVYDSLLALPQDLQAARALCVIALLVALFGLLVYLAGAKCTTCVEEKDSKARLVLTSGIVFVISGVLTLIPVCWTAHAVIRDFYNPLVAEAQKRELGASLYLGWAASGLLLLGGGLLCCTCPSGGSQGPSHYMARYSTSAPAISRGPSEYPTKNYV

SEQ ID NO: 12 - protein, 211 aa, Homo sapiens
MANSGLQLLGFSMALLGWVGLVACTAIPQWQMSSYAGDNIITAQAMYKGLWMDCVTQSTGMMSCKMYDSVLALSAALQATRALMVVSLVLGFLAMFVATMGMKCTRCGGDDKVKKARIAMGGGIIFIVAGLAALVACSWYGHQIVTDFYNPLIPTNIKYEFGPAIFIGWAGSALVILGGALLSCSCPGNESKAGYRAPRSYPKSNSSKEYV

SEQ ID NO: 13 - protein, 225 aa, Homo sapiens
MATHALEIAGLFLGGVGMVGTVAVTVMPQWRVSAFIENNIVVFENFWEGLWMNCVRQANIRMQCKIYDSLLALSPDLQAARGLMCAASVMSFLAFMMAILGMKCTRCTGDNEKVKAHILLTAGIIFIITGMVVLIPVSWVANAIIRDFYNSIVNVAQKRELGEALYLGWTTALVLIVGGALFCCVFCCNEKSSSYRYSIPSHRTTQKSYHTGKKSPSVYSRSQYV

SEQ ID NO: 14 - protein, 217 aa, Homo sapiens
MASTGLELLGMTLAVLGWLGTLVSCALPLWKVTAFIGNSIVVAQVVWEGLWMSCVVQSTGQMQCKVYDSLLALPQDLQAARALCVIALLLALLGLLVAITGAQCTTCVEDEGAKARIVLTAGVILLLAGILVLIPVCWTAHAIIQDFYNPLVAEALKRELGASLYLGWAAAALLMLGGGLLCCTCPPPQVERPRGPRLGYSIPSRSGASGLDKRDYV

SEQ ID NO: 15 - protein, 228 aa, Homo sapiens
MASTASEIIAFMVSISGWVLVSSTLPTDYWKVSTIDGTVITTATYWANLWKACVTDSTGVSNCKDFPSMLALDGYIQACRGLMIAAVSLGFFGSIFALFGMKCTKVGGSDKAKAKIACLAGIVFILSGLCSMTGCSLYANKITTEFFDPLFVEQKYELGAALFIGWAGASLCIIGGVIFCFSISDNNKTPRYTYNGATSVMSSRTKYHGGEDFKTTNPSKQFDKNAYV

SEQ ID NO: 16 - protein, 207 aa, Homo sapiens
MVATCLQVVGFVTSFVGWIGVIVTTSTNDWVVTCGYTIPTCRKLDELGSKGLWADCVMATGLYHCKPLVDILILPGYVQACRALMIAASVLGLPAILLLLTVLPCIRMGQEPGVAKYRRAQLAGVLLILLALCALVATIWFPVCAHRETTIVSFGYSLYAGWIGAVLCLVGGCVILCCAGDAQAFGENRFYYTAGSSSPTHAKSAHV

SEQ ID NO: 17 - protein, 244 aa, Homo sapiens
MGCRDVHAATVLSFLCGIASVAGLFAGTLLPNWRKLRLITFNRNEKNLTVYTGLWVKCARYDGSSDCLMYDTTWYSSVDQLDLRVLQFALPLSMLIAMGALLLCLIGMCNTAFRSSVPNIKLAKCLVNSAGCHLVAGLLFFLAGTVSLSPSIWVIFYNIHLNKKFEPVFSFDYAVYVTIASAGGLFMTSLILFIWYCTCKSLPSPFWQPLYSHPPSMHTYSQPYSARSRLSAIEIDIPVVSHTT

SEQ ID NO: 18 - protein, 239 aa, Homo sapiens
MASTAVQLLGFLLSFLGMVGTLITTILPHWRRTAHVGTNILTAVSYLKGLWMECVWHSTGIYQCQIYRSLLALPQDLQAARALMVISCLLSGIACACAVIGMKCTRCAKGTPAKTTFAILGGTLFILAGLLCMVAVSWTTNDVVQNFYNPLLPSGMKFEIGQALYLGFISSSLSLIGGTLLCLSCQDEAPYRPYQAPPRATTTTANTAPAYQPPAAYKDNRAPSVTSATHSGYRLNDYV

SEQ ID NO: 19 - protein, 228 aa, Homo sapiens
MSMAVETFGFFMATVGLLMLGVTLPNSYWRVSTVHGNVITTNTIFENLWFSCATDSLGVYNCWEFPSMLALSGYIQACRALMITAILLGFLGLLLGIAGLRCTNIGGLELSRKAKLAATAGALHILAGICGMVAISWYAFNITRDFFDPLYPGTKYELGPALYLGWSASLISILGGLCLCSACCCGSDEDPAASARRPYQAPVSVMPVATSDQEGDSSFGKYGRNAYV

SEQ ID NO: 20 - protein, 305 aa, Homo sapiens
MTSRTPLLVTACLYYSYCNSRHLQQGVRKSKRPVFSHCQVPETQKTDTRHLSGARAGVCPCCHPDGLLATMRDLLQYIACFFAFFSAGFLIVATWTDCWMVNADDSLEVSTKCRGLWWECVTNAFDGIRTCDEYDSILAEHPLKLVVTRALMITADILAGFGFLTLLLGLDCVKFLPDEPYIKVRICFVAGATLLIAGTPGIIGSVWYAVDVYVERSTLVLHNIFLGIQYKFGWSCWLGMAGSLGCFLAGAVLTCCLYLFKDVGPERNYPYSLRKAYSAAGVSMAKSYSAPRTETAKMYAVDTRV

SEQ ID NO: 21 - protein, 224 aa, Homo sapiens
MAFYPLQIAGLVLGFLGMVGTLATTLLPQWRVSAFVGSNIIVFERLWEGLWMNCIRQARVRLQCKFYSSLLALPPALETARALMCVAVALSLIALLIGICGMKQVQCTGSNERAKAYLLGTSGVLFILTGIFVLIPVSWTANIIIRDFYNPAIHIGQKRELGAALFLGWASAAVLFIGGGLLCGFCCCNRKKQGYRYPVPGYRVPHTDKRRNTTMLSKTSTSYV

SEQ ID NO: 22 - protein, 261 aa, Homo sapiens
MSTTTCQVVAFLLSILGLAGCIAATGMDMWSTQDLYDNPVTSVFQYEGLWRSCVRQSSGFTECRPYFTILGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 23 - protein, 224 aa, Mus musculus
MANSGLQLLGYFLALGGWVGIIASTALPQWKQSSYAGDAIITAVGLYEGLWMSCASQSTGQVQCKLYDSLLALDGHIQSARALMVVAVLLGFVAMVLSVVGMKCTRVGDSNPTAKSRVAISGGALFLLAGLCTLTAVSWYATLVTQEFFNPSTPVNARYEFGPALFVGWASAGLAMLGGSFLCCTCPEPERANSIPQPYRSGPSTAAREPVVKLPASVKGPLGV

SEQ ID NO: 24 - protein, 219 aa, Homo sapiens
MASAGLQLLAFILALSGVSGVLTATLLPNWKVNVDVDSNIITAIVQLHGLWMDCTWYSTGMFSCALKHSILSLPIHVQAARATMVLACVLSALGICTSTVGMKCTRLGGDRETKSHASFAGGVCFMSAGISSLISTVWYTKEIIANFLDLTVPESNKHEPGGAIYIGFISAMLLFISGMIFCTSCIKRNPEARLDPPTQQPISNTQLENNSTHNLKDYV

SEQ ID NO: 25 - protein, 220 aa, Homo sapiens
MALVFRTVAQLAGVSLSLLGWVLSCLTNYLPHWKNLNLDLNEMENWTMGLWQTCVIQEEVGMQCKDFDSFLALPAELRVSRILMFLSNGLGFLGLLVSGFGLDCLRIGESQRDLKRRLLILGGILSWASGVTALVPVSWVAHKTVQEFWDENVPDFVPRWEFGEALFLGWFAGLSLLLGGCLLHCAACSSHAPLASGHYAVAQTQDHHQELETRNTNLKH

SEQ ID NO: 26 - protein, 292 aa, Homo sapiens
MRTPVVMTLGMVLAPCGLLLNLTGTLAPGWRLVKGFLNQPVDVELYQGLWDMCREQSSRERECGQTDQWGYFEAQPVLVARALMVTSLAATVLGLLLASLGVRCWQDEPNFVLAGLSGVVLFVAGLLGLIPVSWYNHFLGDRDVLPAPASPVTVQVSYSLVLGYLGSCLLLLGGFSLALSFAPWCDERCRRRRKGPSAGPRRSSVSTIQVEWPEPDLAPAIKYYSDGQHRPPPAQHRKPKPKPKVGFPMPRPRPKAYTNSVDVLDGEGWESQDAPSCSTHPCDSSLPCDSDL

SEQ ID NO: 27 - protein, 220 aa, Homo sapiens
MALIFRTAMQSVGLLLSLLGWILSIITTYLPHWKNLNLDLNEMENWTMGLWQTCVIQEEVGMQCKDFDSFLALPAELRVSRILMFLSNGLGFLGLLVSGFGLDCLRIGESQRDLKRRLLILGGILSWASGITALVPVSWVAHKTVQEFWDENVPDFVPRWEFGEALFLGWFAGLSLLLGGCLLNCAACSSHAPLALGHYAVAQMQTQCPYLEDGTADPQV

SEQ ID NO: 28 - protein, 229 aa, Homo sapiens
MAWSFRAKVQLGGLLLSLLGWVCSCVTTILPQWKTLNLELNEMETWIMGIWEVCVDREEVATVCKAFESFLSLPQELQVARILMVASHGLGLLGLLLCSFGSECFQFHRIRWVFKRRLGLLGRTLEASASATTLLPVSWVAHATIQDFWDDSIPDIIPRWEFGGALYLGWAAGIFLALGGLLLIFSACLGKEDVPFPLMAGPTVPLSCAPVEESDGSFHLMLRPRNLVI

(19) United States
(12) Patent Application Publication
(10) Pub. No.: US 2024/0350646 A1
(43) Pub. Date: Oct. 24, 2024

(54) METHODS AND COMPOSITIONS FOR DELIVERY TO AND ACROSS THE BLOOD BRAIN BARRIER

(71) Applicant: NUtech Ventures, Lincoln, NE (US)

(72) Inventors: Forrest KIEVIT, Lincoln, NE (US); Alex VECCHIO, Lincoln, NE (US); Sourav ROY, Lincoln, NE (US); Badrul Alam BONY, Lincoln, NE (US); Aria TARUDJI, Lincoln, NE (US); Punita DHAWAN, Lincoln, NE (US); Saiprasad GOWRIKUMAR, Lincoln, NE (US)

(21) Appl. No.: 18/683,871

(22) PCT Filed: Aug. 19, 2022
(86) PCT No.: PCT/US22/40852

§ 371 Date: Feb. 15, 2024

Related U.S. Application Data
(60) Provisional application No. 63/235,525, filed on Aug. 20, 2021.

(51) Int. Cl. A61K 47/62; A61K 47/10; A61K 47/69; A61K 49/18; B82Y 5/00

(52) U.S. Cl. CPC A61K 47/62; A61K 47/10; A61K 47/6929; A61K 49/186; A61K 49/1866; B82Y 5/00

(57) ABSTRACT

Described herein are nanoparticle compositions that can be used to target specific regions of the blood brain barrier (BBB). Such nanoparticle compositions can be used to deliver therapeutics to or across the BBB or to image the BBB or the permeability thereof.

### CROSS-REFERENCE TO RELATED APPLICATIONS

This application claims the benefit of priority to U.S. Application No. 63/235,525, filed Aug. 20, 2021. The content of the prior application is incorporated herein by reference in its entirety.

### FEDERALLY SPONSORED RESEARCH OR DEVELOPMENT

This invention was made with government support under R01 NS109488 and R35 GM138368 awarded by the National Institutes of Health and under BX002086 awarded by the Department of Veterans Affairs. The government has certain rights in the invention.

### TECHNICAL FIELD

This disclosure generally relates to compositions that can be used for imaging or for therapeutics.

### BACKGROUND

Aging-induced alterations to the blood-brain barrier (BBB) are increasingly being seen as a primary event in chronic progressive neurological disorders that lead to cognitive decline. With the goal of increasing delivery into the brain in hopes of effectively treating these diseases, a large focus has been placed on developing BBB permeable materials. However, these strategies have suffered from lack of specificity towards regions of disease progression.

Therefore, a delivery vehicle that can be targeted to specific regions of the blood brain barrier is desirable.

### SUMMARY

A nanoparticle composition is described that can be used to target specific regions of the blood brain barrier (BBB). Such a nanoparticle composition can be used to deliver therapeutics to or across the BBB or to image the BBB or the permeability thereof.

In one aspect, nanoparticles comprising a claudin or occludin polypeptide or portion thereof conjugated thereto is provided. Typically, the nanoparticle carries a payload.

Representative claudin polypeptides include, without limitation, claudin-1, claudin-3, claudin-5, claudin-11, claudin-12, claudin-25, and claudin-27. In some embodiments, the claudin polypeptide is claudin-1. A representative claudin-1 polypeptide has the amino acid sequence shown in SEQ ID NO:2, and representative fragments of a claudin-1 polypeptide are selected from Cldn146 (SEQ ID NO:3) or Cldn53 (SEQ ID NO:4).

In some embodiments, the payload is an imaging agent. Representative imaging agents include, without limitation, Gd or a radiotracer.

In some embodiments, the payload is a therapeutic agent. Representative therapeutic agents include, without limitation, bispecific antibodies, monoclonal antibodies, immunoglobulin fragments, immunotoxins, nucleic acids, proteins (e.g., recombinant proteins), drugs, small molecules, pharmaceuticals, or combinations thereof.

In some embodiments, the nanoparticle is an ultrasmall nanoparticle (e.g., about 3.5 nm in diameter). In some embodiments, the NPs are coated with PEG diacid.

In another aspect, methods of delivering a therapeutic to or across the blood brain barrier (BBB) in an individual are provided. Such methods typically include delivering a nanoparticle as described herein to the individual, wherein the payload is a therapeutic.

In still another aspect, methods of imaging the blood brain barrier or determining the permeability of the blood brain barrier in an individual are provided. Such methods typically include administering a nanoparticle as described herein to the individual, wherein the payload is an imaging agent.

In some embodiments, the individual is experiencing neurocognitive decline or is at risk of experiencing neurocognitive decline. In some embodiments, the individual has or has had a brain tumor or a traumatic brain injury (TBI). In some embodiments, the individual has been diagnosed with a neurological disorder (e.g., Alzheimer's disease).

In some embodiments, the administering step is oral administration.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1A-1F are results from experiments characterizing NPs. FIG. 1A is HRTEM images of the PEG diacid coated ultrasmall Gd nanoparticles. The circles show the diameter of the nanoparticle, which is 3.5 nm. FIG. 1B is a graph from DLS, showing the average size of the nanoparticle is 10.5 nm. FIG. 1C is a graph showing that the zeta potential value is −16.1 mV for Gd nanoparticles. FIG. 1D is a graph showing the FT-IR absorption spectra of (I) free PEG diacid (red line) and (II) PEG diacid coated ultrasmall Gd nanoparticles (black line). FIG. 1E is a graph showing R1 relaxivity and T1 map images. FIG. 1F is a graph showing R2, and T2 map images of Gd nanoparticles.

FIG. 2A-2B are results from experiments showing NP accumulation in the corpus callosum. FIG. 2A are K^{trans} maps showing NPs uptake in corpus callosum. FIG. 2B is a graph showing mean K^{trans} values of different peptide conjugated NPs in corpus callosum. Data are presented as an average of mice (3-8 mice) experiments with standard deviations, *p<0.05.

FIG. 3A-3B are results from experiments showing NP accumulation in the hippocampus. FIG. 3A are K^{trans} maps showing NPs uptake in hippocampus. FIG. 3B is a graph showing mean K^{tr} values of different peptide conjugated NPs in hippocampus. Data are presented as an average of mice (3-8 mice) experiments with standard deviations, *p<0.05.

FIG. 4A-4F are the results of experiments showing that immunofluorescence staining for claudin-1 (green) in the corpus callosum and hippocampus correlates with the K^{trans} value of the NP. Blue DAPI staining indicates nuclei. Scale bar is 20 μm. FIG. 4A are representative images of the corpus callosum of the old and young mice, as well as the high and low K^{trans} value mice. FIG. 4B is a linear regression graph between K^{trans} and integrated density of claudin-1 fluorescence signal in the corpus callosum. FIG. 4C are representative images of the CA1, CA3, and dentate gyrus regions of the old and young mice, as well as the high and low K^{trans} value mice. FIGS. 4D, 4E and 4F are linear regression graphs between K^{trans} and integrated density of claudin-1 fluorescence signal in the CA1, CA3, and dentate gyrus, respectively. One-tailed Pearson correlation coefficient showed high correlation between integrated density and K^{trans} value in the corpus callosum, CA1, CA2/3, and dentate gyrus regions (r=0.6622, 0.8826, 0.9039, and 0.8487 with p=0.037, 0.002, 0.001, and 0.004, respectively).

FIG. 5A-5D demonstrate that C1C2-NP specifically binds to both mouse and human Claudin-1, and FIG. 5A-5C are results based on in vitro immunofluorescence of NPs binding claudin-1 in mouse brain endothelial (bEnd.3) cells. FIG. 5A is Western blot analysis of claudin-1 in bEnd.3 cells and those induced to express claudin-1 through ethanol exposure or exogenous expression through plasmid transfection. FIG. 5B is a graph of the quantification of claudin-1 expression relative to β-actin. *** indicates a statistical difference (p<0.0001) in claudin-1 expression as compared to control cells. FIG. 5C are images that show C1C2-NP (magenta) binding to ethanol induced mouse claudin-1 and exogenously expressed human claudin-1. Scale bar represents 10 μm. FIG. 5D shows the dissociation constants (K_{D}) for C1C2, C1C2-NP, and NP complex formation with human claudin-1. Binding curves for the complex formation between human claudin 1 and C1C2-NP (green), NP (red), and C1C2 peptide (blue) using microscale thermophoresis. The concentration of human claudin 1 was varied from 74 μM-0.2 nM in all three experiments. The concentrations of C1C2-NP, NP, and peptide were fixed at 70 nM, 2 μM and 1 μM, respectively. The buffer used for these measurements contained 10 mM Tris HCl, pH 8.0, 100 mM NaCl, 4% glycerol and 0.04% b-DDM. The experimental data points with human claudin 1 and C1C2-NP are reported as standard deviations from two independent measurements. The estimated value of the dissociation constant is 21±14 μM. Human claudin 1 does not show significant binding to the non-targeted NP, as the dissociation constant is very high >300 μM. The data with the peptide alone does not fit at all, indicating little binding with free peptide in solution.

FIG. 6 are images showing in vivo C1C2-NP (red) accumulation in the corpus callosum and hippocampus co-localize with the immunofluorescence staining for claudin-1 (green). Blue DAPI staining indicates nuclei. Scale bar is 20 μm. NP accumulation can be observed in the old mice with high K^{trans} value, but not in the young mice with low K^{trans} value.

FIG. 7A are K^{trans} maps showing NP uptake in cortex (CTX).

FIG. 7B is a graph showing data presented as an average of mice (3-8 mice) experiments with standard deviations, *p<0.05

FIG. 8A are K^{trans} maps showing NP uptake in hypothalamus (HT).

FIG. 8B is a graph showing data presented as an average of mice (3-8 mice) experiments with standard deviations, *p<0.05

FIG. 9A are K^{trans} maps showing NP uptake in muscles.

FIG. 9B is a graph showing data presented as an average of mice (3-8 mice) experiments with standard deviations, *p<0.05

FIG. 10A-10D are results from experiments demonstrating that C1C2-NP shows little specific binding to other claudins. FIG. 10A is a graph showing binding of human claudins 3, 4 and 9 to C1C2-NPs using microscale thermophoresis. FIG. 10B is a table showing that fitting of the data points in the case of human claudins 3 and 4 resulted in undefined 95% confidence intervals and, in the case of human claudin 9, the data did not fit even after the maximum number of iterations with the sigmoidal dose response curve. FIG. 10C is a graph showing the binding of human claudins 4 and 9 to control NPs monitored using microscale thermophoresis. FIG. 10D is a table showing that fitting of the data points in the case of human claudin 4 resulted in undefined 95% confidence intervals, while in the case of human claudin 9, the estimated binding constant with control NPs was ˜3 μM. Data fitting for human claudins 3, 4 and 9 were performed using Graph Pad Prism.

FIG. 11 is an alignment of claudins (SEQ ID NO:2 and 7-28 (top to bottom)). All available human claudin sequences, as well as those of murine mCLDN-15 and -19, were aligned using T-Coffee. Carrots (\hat{ }) identify amino acids utilized in disulfide bond formation; plus signs (+) identify amino acids utilized in HCV entry; and asterisks (*) identify amino acids utilized in cis interactions. Boxes indicate C residue positions in non-conserved regions of the sequence.

### DETAILED DESCRIPTION

The blood-brain barrier (BBB) plays a crucial role in the central nervous system (CNS) by creating a barrier between the neural tissue and the blood to prevent contamination from foreign substances. Aging is linked with decreased BBB integrity as well as functional impairment of transporters and can aggravate BBB responses to any CNS injury and systemic inflammatory stimuli across multiple different brain regions including the cortex, hippocampus, and corpus callosum. This is a result of age-induced alterations in gene expression, mitochondrial dysfunction, or abnormal protein accumulation in the CNS. Moreover, these alterations have been implicated in promoting neurodegeneration, declined cognitive function, reduced cerebral blood flow, and vasculopathies; thus, BBB disruption can initiate during normal aging and lead to mild cognitive impairment and progression to Alzheimer's disease and other dementias. In fact, BBB breakdown has recently been proposed as an early biomarker of neurocognitive decline.

The function of the BBB is controlled through a complex interaction with the neurovascular unit (NVU), but BBB function relies on adequate assembly of tight junction (TJ) proteins on the surface of brain endothelial cells. Transmembrane proteins play a vital role in the formation and maintenance of TJ function. TJ transmembrane protein members at the BBB include a number of claudins (e.g., claudin-1, claudin-3, claudin-5, claudin-11, claudin-12, claudin-25, and claudin-27) and occludin. See, e.g., Bemdt et al. (2019, Cell. Mol. Life Sci., 76(10):1987-2002). Claudins and occludin have four membrane-spanning helices and two extracellular segments that facilitate interactions between other TJ proteins to ultimately form TJ barriers. TJ protein expression is altered during aging and leads to the observed age-related leakiness of the BBB. For example, it has been shown that claudin-5 expression decreases during aging and disease and that brain endothelial cell surface expression of claudin-1 increases during aging and in response to injury, resulting in increased leakiness of the BBB. It has been proposed, therefore, that claudin-1 may impair the function of claudin-5 at the BBB through disruption of claudin-5 interactions with other TJ proteins. Therefore, claudins (e.g., claudin-1, claudin-3, claudin-5, claudin-11, claudin-12, claudin-25, and claudin-27) as well as occludin represent cell surface receptor candidates for specific targeting of the BBB (e.g., prior to or during aging, prior to or during neurocognitive decline).

Nanoparticles (NPs) offer the advantage of multi-functionality whereby they can be engineered to display a targeting agent on their surface to promote binding to specific cells, provide contrast in various imaging modalities, and carry a therapeutic payload to increase target engagement. There has been significant effort placed on developing NPs that can cross the BBB to improve brain delivery for various neurological disorders. Strategies typically involve passive strategies that rely on increased leakiness of the BBB such as with a brain tumor or following trauma, or through active strategies with the so-called “trojan horse” method that exploits transporters expressed on brain endothelial cells to hijack the native transport mechanism into the brain. These include targets such as the transferrin transporters, amino acid transporters, and glucose transporters. However, this strategy suffers from the lack of specificity to target diseased brain regions as these transporters are typically ubiquitously expressed. Furthermore, the transporters can become downregulated during the disease process, thus reducing uptake in regions where delivery is desired.

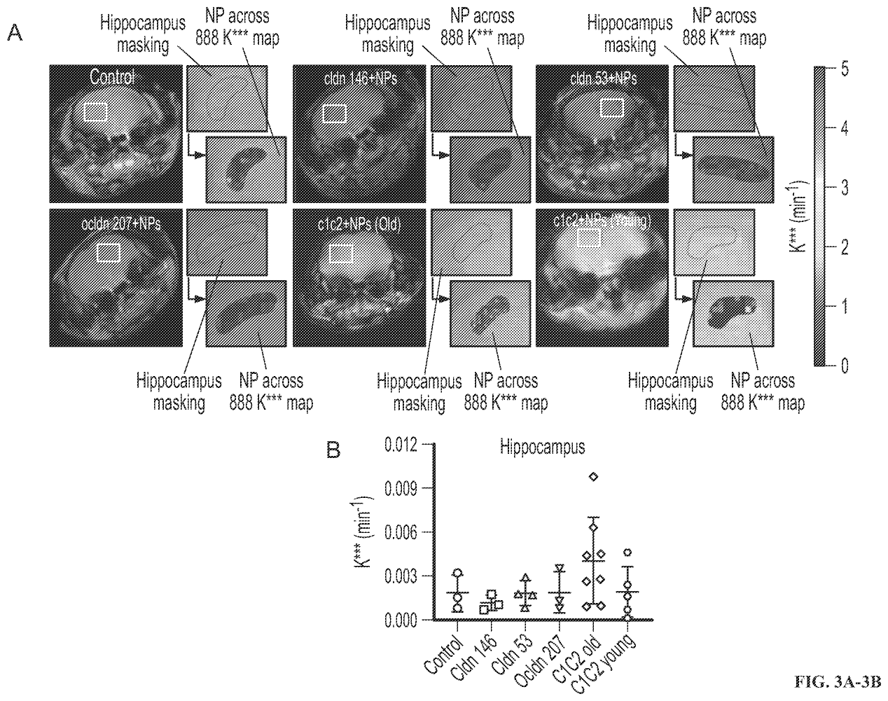

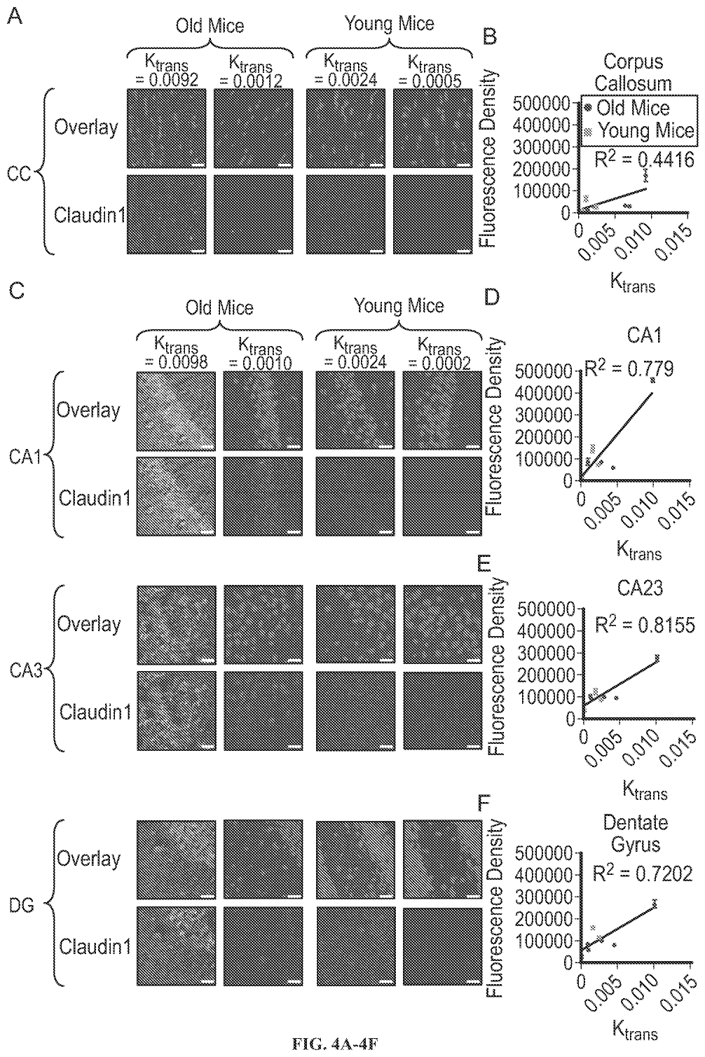

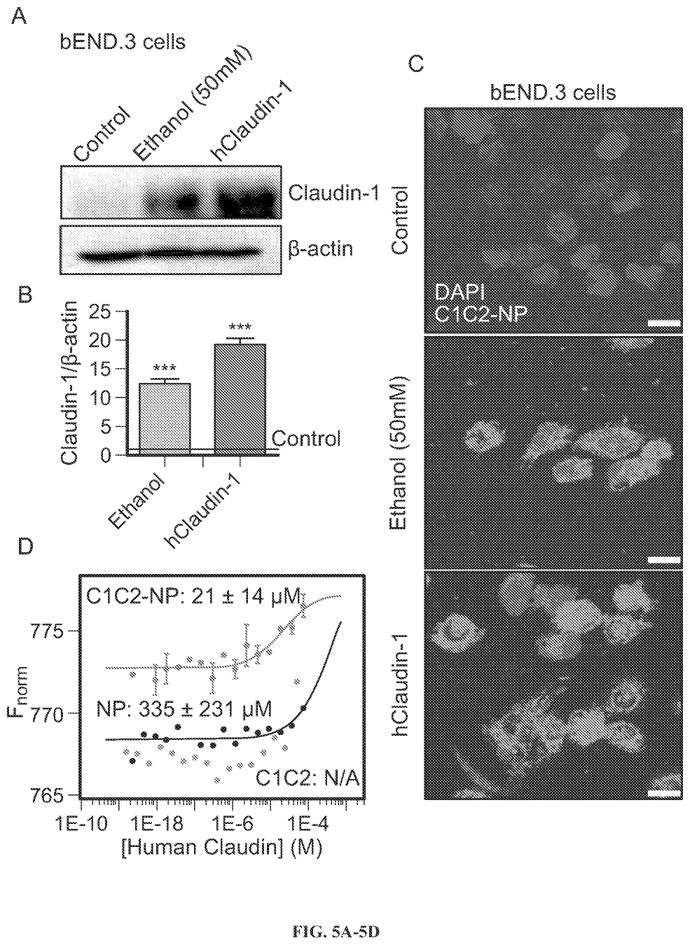

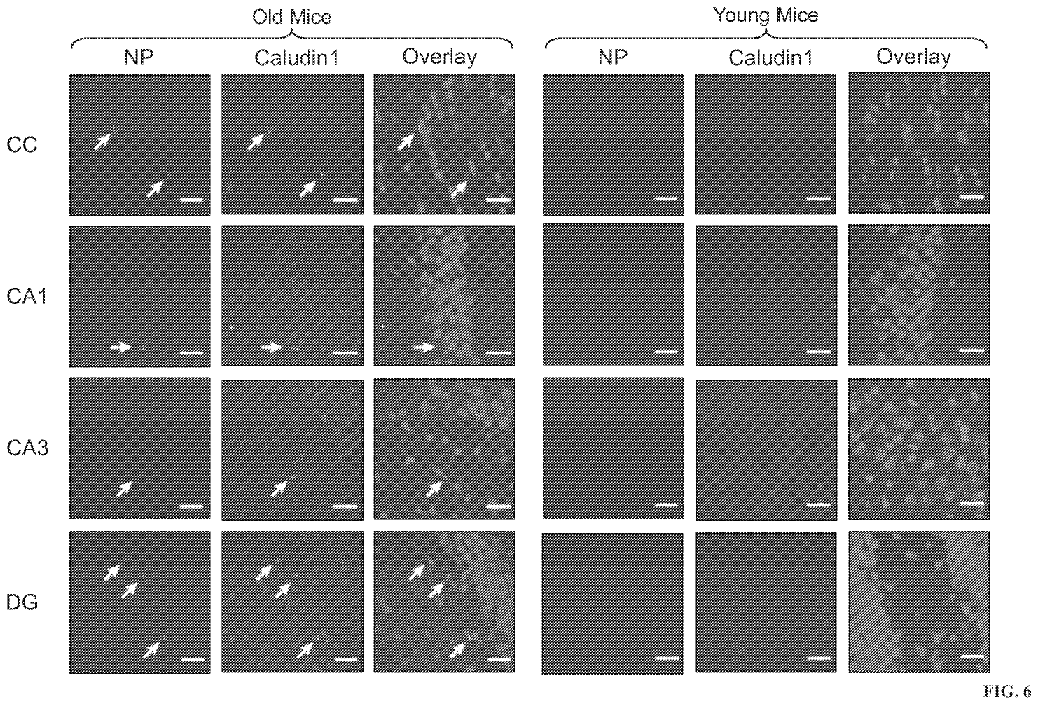

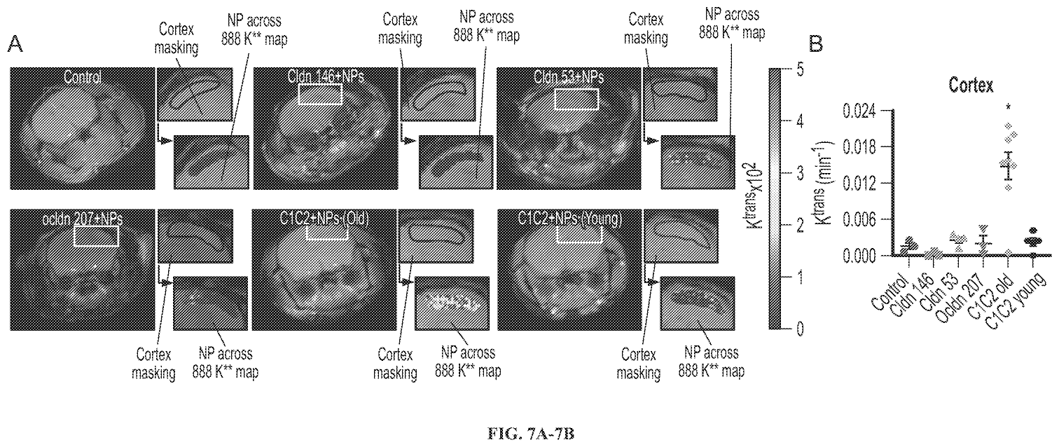

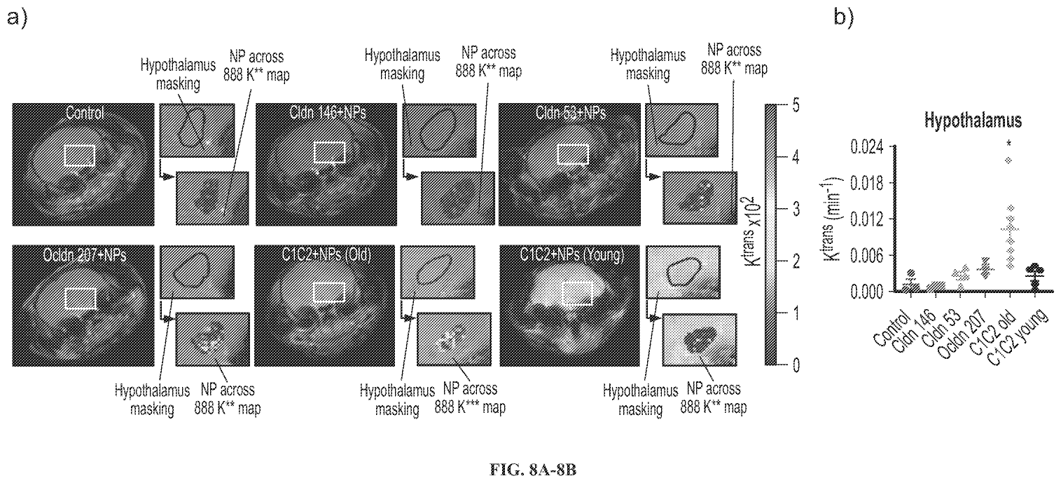

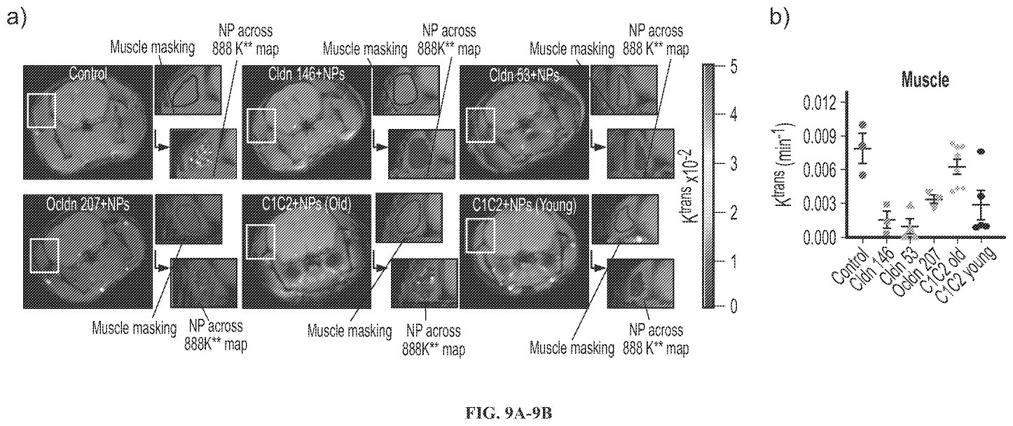

Here, ultrasmall 3.5 nm Gd NPs labeled with AF647 were utilized to provide multimodal imaging through MRI and fluorescence. BBB permeability is typically investigated utilizing small molecule Gd-based contrast agents for DCE-MRI. We have recently extended the use of this DCE-MRI method for use with contrast-enhancing NPs to compare the permeabilities of different NPs. The significantly higher K^{trans} observed in the corpus callosum using C1C2 NPs compared with the control non-targeted NPs (FIG. 2) indicates significantly higher accumulation and retention when claudin-1 is targeted with the C1C2 peptide as compared to other peptides and peptides against occludin. This result is in accordance with previous work that found, following BBB disruption, occludin and claudin-5 in the brain endothelium are no longer present and instead move away and colocalize with astrocytes. There are trending differences in the hippocampus but without significance (FIG. 3), but K^{trans} in the corpus callosum and hippocampus is directly correlated with claudin-1 expression in these regions (FIG. 4), suggesting higher biological variability in claudin-1 expression in the hippocampus as compared to the corpus callosum. These findings of limited accumulation in young mice support previous work showing that BBB leakiness is lower in younger adults.

The presence of increased claudin-1 on the BBB is correlated with reduced BBB function, although the specific role of claudin-1 in the normal function of the BBB is still under investigation. The question of whether increased claudin-1 cell surface expression is utilized in BBB repair following injury or disease or if increased claudin-1 expression itself leads to increased BBB leakiness requires further investigation. Claudin-1 may be required for embryonic development of the BBB and replaced by claudin-5 upon complete maturation of the BBB as claudin-1 is involved in neural tube closure in chick embryos. However, Tran et al., found that β-catenin activates the claudin-1 promoter and that inducible knockout of β-catenin in mice resulted in reduced claudin-1 expression and increased BBB leakiness even though claudin-5 mRNA levels were not affected, which suggests claudin-1 may have a role in maintaining BBB integrity. Nevertheless, these results may be affected by other mechanisms by which β-catenin helps maintain BBB integrity as Leibner et al., showed that claudin-1 expression is not under control of Wnt/β-catenin signaling. This idea is bolstered by Sladojevic et al., who found that, following injury to the BBB caused by stroke, increased claudin-1 expression correlated with reduced repair and increased leakiness of the BBB and that decreasing cell surface expression of claudin-1 resulted in improved BBB repair. The detrimental effects of claudin-1 were thought to be caused by claudin-1-zona occludin and claudin-1-claudin-5 interactions that reduced claudin-5-mediated TJ integrity. Conversely, Pfeiffer et al. found that increased claudin-1 expression correlated with improved BBB integrity following autoimmune encephalomyelitis injury. It is thought that claudin-1 is stored in intracellular microvessels that quickly traffic to the cell membrane following injury to promote sealing of the BBB. This claudin-1 would then eventually be replaced by de novo synthesis of claudin-5 to complete regeneration of the BBB. Combined, this suggests claudin-1 may act as an initial scaffold for subsequent complete sealing of the TJ by claudin-5 in the formation of the BBB. In aging or disease, however, chronic cell surface expression of claudin-1 may be an early event in BBB disruption and chronic progression of BBB dysfunction by chronically inhibiting normal claudin-5 interactions that would completely seal the BBB. Our results in young and aged mice suggest abnormal claudin-1 cell surface expression at the BBB is an age-related event that can be actively targeted by NPs, both as a tool to study age related BBB dysfunction and as a delivery vehicle for site specific delivery of therapeutics.

C1C2 peptide has been shown to promote resealing of the BBB. C1C2 binds to the first extracellular loop of claudin-1, which weakens trans- and cis-claudin-1 interactions and reduces claudin-1/ZO-1 interactions. Similarly, we showed C1C2-NP actively bind to both mouse and human claudin-1 in brain endothelial cells induced to express claudin-1 through ethanol exposure or exogenously express human claudin-1 through plasmid transfection (FIG. 5). Furthermore, the multivalent effect of multiple C1C2 peptides on the surface of the C1C2-NP resulted in a high binding affinity to claudin-1 with a dissociation constant of 21 μM. Previous reports have indicated that the permeability of the tight junctions in Caco II and MDCK 11 cell lines can be changed using C1C2 peptide at ˜200 μM. This corresponds to saturating concentrations of the peptide with respect to the K_{d}. Therefore, both the multivalency effect as well as the anchoring of the C1C2 peptide to the surface of a solid NP may help increase the binding affinity towards claudin-1.

Therefore, since claudin-1/claudin-5 are considered incompatible, reducing this interaction, for example, may allow claudin-5 interactions to predominate and result in tighter BBB sealing. NP-bound C1C2 may further promote claudin-1 internalization from the increased wrapping energy induced by the NP core. This could then lead to increased cytosolic degradation of claudin-1. Thus, specific delivery into the endothelial cells with abnormal TJ protein expression would make for an ideal treatment strategy if the NPs were modified to also deliver a therapeutic that was shown to normalize TJ protein expression.

In summary, BBB integrity is central to maintaining brain health and aging-induced alterations in TJ protein expression can lead to chronic leakiness of the BBB, which is directly correlated with cognitive impairment. With the goal of establishing a targeting method for promoting NP delivery specifically to regions of the brain with altered function, a claudin-1 targeted NP (C1C2-NP) was developed to target this TJ protein that appears to be involved in the chronic impairment of BBB integrity. We find that C1C2-NP has high accumulation and retention in brain regions that have shown age-induced increases in BBB leakiness. Furthermore, C1C2-NP accumulation and retention are directly correlated with claudin-1 expression regardless of mouse age. Overall, our findings support the idea of increased claudin-1 cell surface expression observed during normal aging and that these regions of altered expression can be targeted with a NP. Therefore, these NPs offer utility as a tool to monitor alterations in TJ protein expression (e.g., claudins or occludin) that may cause BBB leakiness through non-invasive MR imaging as well as a targeted delivery vehicle to improve site-specific target engagement of delivered therapeutics.

### Nanoparticles

Essentially any material that can be made nanosized (typically 1-200-300 nm) can be used to produce a nanoparticle, which, in turn, can be used to deliver a claudin polypeptide. Representative materials that can be nanosized and, hence, used to make nanoparticles, include, without limitation, silver, gold, hydroxyapatite, clay, titanium dioxide, silicon dioxide, zirconium dioxide, carbon, diamond, aluminium oxide, chitosan, alginate, xanthan gum, cellulose, liposomes, and micelles. See, e.g., Salata, 2004, J. Nanobiotech., 2(1):3, for a description of a number of different materials suitable for making nanoparticles.

In order to be able to deliver a claudin polypeptide, the material must have a functional group that can be used to chemically attach a claudin polypeptide; such a functional group can be attached directly to the material or can be attached to a coating that is applied to the material or the nanoparticle. It would be appreciated that there are a number of different chemistries that can be used to attach the claudin polypeptide to a nanoparticle. For example, a claudin polypeptide can be attached to a nanoparticle using EDC/NHS chemistry (e.g., 1-Ethyl-3-[3-dimethylaminopropyl]-carbodiimide hydrochloride, a zero-length cross-linking agent, used to couple carboxyl groups to primary amines, typically via the formation of amine-reactive NHS-esters). EDC/NHS chemistry, as well as a number of other chemistries suitable for attaching a claudin polypeptide to a nanoparticle are described in, for example, Hermanson, 2013, “Bioconjugate Techniques,” Academic Press.

### Claudin

Claudins function as major constituents of the tight junction complexes that regulate the permeability of epithelia. While some claudin family members play essential roles in the formation of impermeable barriers, others mediate the permeability to ions and small molecules. Often, several claudin family members are co-expressed and interact with each other, and this determines the overall permeability.

For example, CLDN1 is required to prevent the diffusion of small molecules through tight junctions in the epidermis and is required for the normal barrier function of the skin. The claudin-1 nucleic acid and amino acid sequences are known in the art and can be found, for example, in GenBank Accession Nos. NM_021101.5 (SEQ ID NO: 1) and NP_066924.1 (SEQ ID NO: 2), respectively. It would be appreciated that it would not be necessary to use the entire sequence of claudin-1, and that a portion of claudin-1 (e.g., a fragment) could be conjugated to a nanoparticle as described herein. Suitable fragments are those that sufficiently target the nanoparticles to the BBB.

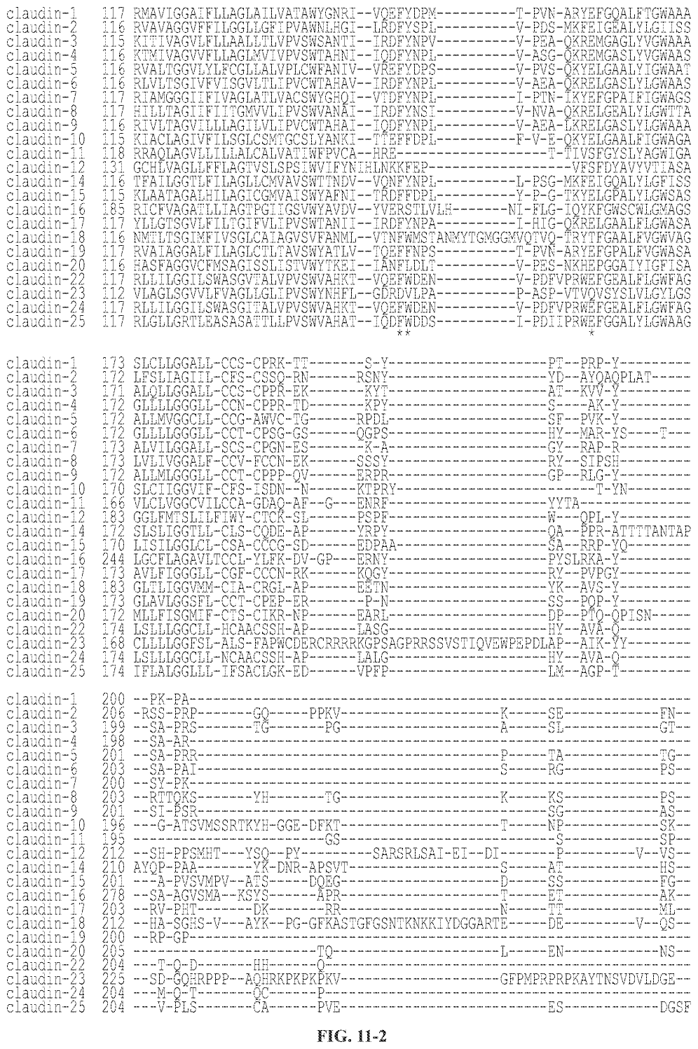

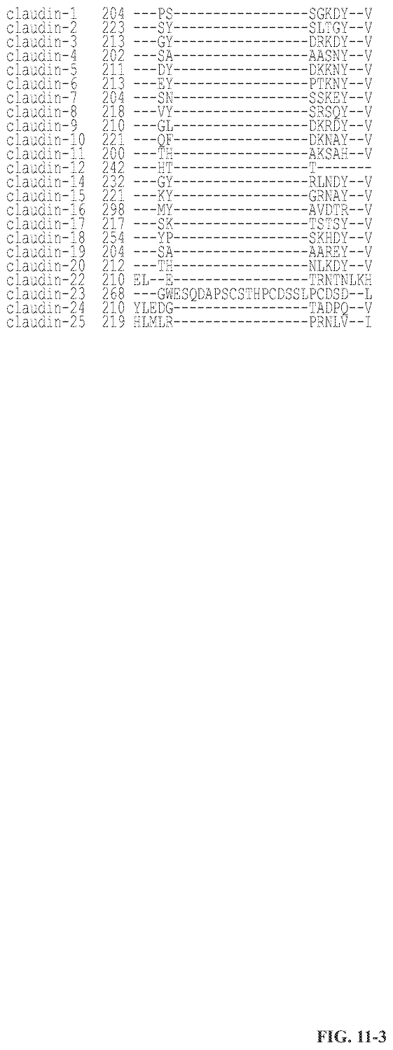

Similarly, any number of other claudin sequences or a portion thereof (e.g., a fragment) can be used in the compositions and methods described herein. By way of example, an alignment of the available human claudin amino acid sequences are shown in FIG. 11. Representative human claudin sequences are shown in GenBank Accession No. NP_001297.1 (claudin-3); AAH19290.2 (claudin-5); NP 005593.2 (claudin-11); NP_001172002.1 (claudin-12); and NP_001094859.1 (claudin-25). In addition, a representative human occludin sequence can be found in GenBank Accession No. BAG70120.1.

In addition to the human claudin and occludin nucleic acids and polypeptides disclosed herein, the skilled artisan will further appreciate that changes can be introduced into the claudin nucleic acid molecule, thereby leading to changes in the amino acid sequence of the encoded claudin polypeptide. For example, changes can be introduced into nucleic acid coding sequences using mutagenesis (e.g., site-directed mutagenesis, PCR-mediated mutagenesis) or by chemically synthesizing a nucleic acid molecule having such changes. Such nucleic acid changes can lead to conservative and/or non-conservative amino acid substitutions at one or more amino acid residues. A “conservative amino acid substitution” is one in which one amino acid residue is replaced with a different amino acid residue having a similar side chain (see, for example, Dayhoff et al. (1978, in Atlas of Protein Sequence and Structure, 5(Suppl. 3):345-352), which provides frequency tables for amino acid substitutions), and a non-conservative substitution is one in which an amino acid residue is replaced with an amino acid residue that does not have a similar side chain.

Change can be introduced into a human claudin nucleic acid sequence such that the encoded polypeptide differs from the human claudin polypeptide. For example, nucleic acids and polypeptides that differ in sequence from any of SEQ ID NOs: 1 and SEQ ID NOs: 2 and 7-28 can have at least 80% sequence identity (e.g., at least 85%, 90%, 95%, or 99% sequence identity) to SEQ ID NOs: 1 and SEQ ID NOs: 2 and 7-28, respectively.

In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region.

The alignment of two or more sequences to determine percent sequence identity can be performed using the algorithm described by Altschul et al. (1997, Nucleic Acids Res., 25:3389 3402) as incorporated into BLAST (Basic Local Alignment Search Tool) programs, available at ncbi.nlm.nih.gov on the World Wide Web. BLASTN is the program used to align and compare the identity between nucleic acid sequences, while BLASTP is the program used to align and compare the identity between amino acid sequences. When utilizing BLAST programs to calculate the percent identity between a sequence and another sequence, the default parameters of the respective programs generally are used.

### Imaging Agents

The payload described herein can be an imaging agent. A number of imaging agents can be used to visualize cells and cellular activity, such as the chemical processes involved in metabolism, oxygen use or blood flow. The particular imaging agent used generally depends on the method by which the imaging is being obtained and the goals for the imaging.

There are a number of imaging agents known in the art, and new ones are continually being developed. Imaging agents include, without limitation, radiotracers (e.g., radioactive atoms, isotopes), radiocontrast agents (e.g., iodine, barium sulfate), contrast agents (e.g., gadolinium (Ga)-based agents (e.g., Ga(III)), iron oxide agents, iron platinum agents, manganese), and fluorescent/luminescent materials.

Those imaging agents that are amendable to being encapsulated within a nanoparticle are known in the art or can be routinely determined by a person of skill in the art.

### Therapeutics

The payload described herein can be a therapeutic from any number of classes. For example, therapeutics can be bispecific antibodies, monoclonal antibodies, immunoglobulin fragments, immunotoxins, nucleic acids, proteins (e.g., recombinant proteins), drugs, small molecules, pharmaceuticals, or combinations thereof.

A therapeutic can be administered in an effective amount to an individual suffering from a neurological disorder or suffering from neurological decline. Typically, an effective amount is the amount that results in amelioration of symptoms or a prolongation of survival in an individual without inducing any adverse effects. Toxicity and therapeutic efficacy of a therapeutic can be determined by standard pharmaceutical procedures in cell cultures or experimental animals, e.g., by determining the LD₅₀ (the dose lethal to 50% of the population) and the ED₅₀ (the dose therapeutically effective in 50% of the population). The dose ratio of toxic to therapeutic effects is the therapeutic index, which can be expressed as the ratio LD₅₀/ED₅₀. A therapeutic compound typically exhibits a high therapeutic index.

### Administration

Nanoparticles as described herein can be formulated with a pharmaceutically acceptable carrier for delivery to an individual in an effective amount. The particular formulation and the effective amount will be dependent upon a variety of factors including route of administration, dosage and dosage interval of a compound the sex, age, and weight of the individual being treated, the severity of the affliction, and the judgment of the individual's physician.

As used herein, “pharmaceutically acceptable carrier” is intended to include any and all excipients, solvents, dispersion media, coatings, antibacterial and anti-fungal agents, isotonic and absorption delaying agents, and the like, compatible with administration. The use of such media and agents for pharmaceutically acceptable carriers is well known in the art. Except insofar as any conventional media or agent is incompatible with a compound, use thereof is contemplated.

Pharmaceutically acceptable carriers for delivering compounds are well known in the art. See, for example Remington: The Science and Practice of Pharmacy, University of the Sciences in Philadelphia, Ed., 21st Edition, 2005, Lippincott Williams & Wilkins; and The Pharmacological Basis of Therapeutics, Goodman and Gilman, Eds., 12th Ed., 2001, McGraw-Hill Co. The type of pharmaceutically acceptable carrier used in a particular formulation can depend on various factors, such as, for example, the physical and chemical properties of the compound, the route of administration, and the manufacturing procedure.

Pharmaceutically acceptable carriers are available in the art, and include those listed in various pharmacopoeias. See, for example, the U.S. Pharmacopeia (USP), Japanese Pharmacopoeia (JP), European Pharmacopoeia (EP), and British pharmacopeia (BP); the U.S. Food and Drug Administration (FDA) Center for Drug Evaluation and Research (CDER) publications (e.g., Inactive Ingredient Guide (1996)); and Ash and Ash, Eds. (2002) Handbook of Pharmaceutical Additives, Synapse Information Resources, Inc., Endicott, NY.

A pharmaceutical composition that includes a nanoparticle as described herein is typically formulated to be compatible with its intended route of administration. Suitable routes of administration include, for example, oral, rectal, topical, nasal, pulmonary, ocular, intestinal, and parenteral administration. Routes for parenteral administration include intravenous, intramuscular, and subcutaneous administration, as well as intraperitoneal, intra-arterial, intra-articular, intracardiac, intracisternal, intradermal, intralesional, intraocular, intrapleural, intrathecal, intrauterine, and intraventricular administration.

### Neurological Disorders/Neurocognitive Decline

The methods described herein can be useful in the treatment of a large number of neurological disorders. Representative neurological disorders include, without limitation, Alzheimer's disease, amyotrophic lateral sclerosis (ALS), Bell's palsy, epilepsy and other seizure disorders, brain tumors, meningitis, stroke, headaches (cluster, migraine, tension). The methods described herein also can be useful in the treatment of the neurological decline that is observed in the early stages of many neurological disorders.

As used herein, the term “treat,” “treating” or “treatment” of a disease or disorder refers to ameliorating the disease or disorder (e.g., slowing or arresting or reducing the development of the disease or at least one of the clinical symptoms thereof). In some instances, “treat,” “treating” or “treatment” refers to alleviating or ameliorating at least one physical parameter including those which may not be discernible by an individual. In some instances, “treat,” “treating” or “treatment” refers to modulating the disease or disorder, either physically, (e.g., stabilization of a discernible symptom), physiologically, (e.g., stabilization of a physical parameter), or both. In yet another embodiment, “treat,” “treating” or “treatment” refers to preventing or delaying the onset or development or progression of the disease or disorder.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1—Materials

Gadolinium chloride hydrate (GdCl₃·xH₂O, 99.9%), europium (III) nitrate hydrate (Eu(NO₃)₃·5H₂O, 99.9%), triethylene glycol (TEG, 99%), sodium hydroxide (NaOH), Poly(ethylene glycol) diacid (Mn 600), ethyl-3-(3-dimethylaminopropyl)-carbodiimide hydrochloride (EDC, ≥98%), and N-hydroxysuccinimide (NHS, 98%), molecular weight cutoff dialysis membranes (Flot-A-Lyzer, 20 kDa) were purchased from Sigma-Aldrich. Alexa Fluor 647 cadaverine (AF647, Mn˜1000) was purchased from Thermo Fisher Scientific. Cldn1-146⁶³, cldn1-53⁶³, occludin-207⁶⁴ and C1C2²⁰ were purchased from Genscript Corporation, and they are all amidated at the C-terminal and acetylated at the N-terminal. Deionized water in the experiments was obtained by using a Millipore water purification system. All other chemicals and solvents used in this work were high-performance liquid chromatography (HPLC)-grade.

### Example 2—Synthesis of Gd NPs

1 mmol of GdCl₃·xH₂O was added into 30 mL of triethylene glycol containing 100 mL three-necked flask. The mixture was heated to 80° C. and magnetically stirred until the precursors were completely dissolved in the solvent. Then, 3 mmol NaOH was added and continued stirring for 4 h at 180° C. To coat the hydrophobic NPs, 2 mmol PEG-diacid was added and continued the reaction with stirring for 12 h at 150° C. After completely cooling, the synthesized nanoparticles were washed 3 times using deionized water.

### Example 3—Conjugation of NPs

To conjugate NPs to the peptide, 1 mL of 0.1 mg/mL NPs were taken in 0.9 mL PBS. To the NPs 4 mg of EDC and 2 mg of NHS were added in a stepwise fashion with continuous stirring. Peptide (0.1 mg/mL) was then added and continuously stirred for 2 h. A float-a-lyzer dialysis kit was used to remove unconjugated peptides. The whole experiment was conducted in the dark.

To make the NPs fluorescent for microscale thermophoresis measurements, AF647 was attached to the Gd NPs using the EDC-NHS coupling reaction. Briefly, 0.1 mL of 1 mg/mL Gd NPs were taken into 0.9 mL PBS. Then 2 mg of EDC and 1 mg of NHS were added in a stepwise fashion with continuous stirring. Alexa Fluor 647 (0.1 mg/mL) was then added, and the mixture was continuously stirred for 2 h. Unconjugated fluorophore was removed with a Float-a-lyzer dialysis kit. The whole experiment was conducted in the dark.

### Example 4—Characterization

A high voltage transmission electron microscope (TEM) (Tecnai Osiris™, 200 kV) was used to measure particle diameters of PEG diacid coated Gd NPs. A copper grid (PELCO mesh size 400, TED PELLA, INC.) covered with an amorphous carbon membrane was placed onto a filter paper. Then, a sample solution diluted in triply distilled water was dropped over the copper grid by using a micropipette (Eppendorf, 2-20 μL). Dynamic light scattering (DLS) studies of the NPs were conducted using a Malvern Instruments Zetasizer Nano series instrument. Solutions of the NPs were prepared in DPBS (pH 7.4) at a concentration of 0.05 mM. The resulting solutions were filtered with 0.22 μm filters before the measurement. The NP concentration was determined by using an inductively coupled plasma mass spectrometer (ICPMS) (Agilent 7500 cx). To determine this, ˜0.5 mL of the NP solution was taken out and treated with HNO₃ to dissolve nanoparticles in the solution completely. A Fourier transform-infrared (FT-IR) (Nicolet AVATAR 380 FT-IR) was used to verify the surface coating. To record the FT-IR absorption spectrum (400-4000 cm⁻¹), the powder sample was prepared. Peptide attachment to NPs was confirmed using BCA assay. Attaching efficiency was confirmed using the equation, Conjugation Efficiency of peptide (%)=Amount of peptide in NPs/Initial amount of peptide×100.

To calculate the number of peptides per NP, the ratio of molarities of peptides to NP cores was determined. Molarity of the peptides were determined from the conjugation efficiency and molecular weight of the peptides (cldn146, cldn53, ocldn207 and C1C2 is 1897.06, 3027.4, 1978.22 and 2891.12 g/mol, respectively). To calculate the molarity of the NP, the mass of a NP was calculated, where m=ρ×V; where ρ is the density of the NP (7.4 g/cm³ for Gd oxide) and V is the calculated volume of the NP (V=4/3πr³, where r is the radius of the NP). The number of NP in colloidal solution is N=C/m, where C is the concentration of metal in NP solution (0.08 mg/mL) measured by ICP-MS and m is the mass of a NP calculated above. The molarity of NP is M_{NP}=N/V×1/N_{A}, where N is the number of particles calculated before, V is the volume of NP solution and N_{A} is Avogadro's number.

### Example 5—R1 and R2 Relaxivity and R1 and R2 Map Image Measurements

Both R1 and R2 map images, as well as both T1 and T2 relaxation times, were measured by using a 9.4 T MRI instrument (Varian 9.4 T) equipped with a 4 cm Millipede RF imaging probe with triple-axis gradients (100 G/cm max). A series of five aqueous solutions of different concentrations (1.0, 0.5, 0.25, 0.125, 0.0625, and 0.03125 mM Gd) were prepared by diluting each MRI solution with PBS. Then, both map images and relaxation times were measured by using these solutions. The R1 and R2 relaxivities were then estimated from the slopes in the plots of 1/T1 and 1/T2 versus NPs concentration, respectively. The measurement parameters for the fast spin-echo T1 mapping sequence were as follows: the external MR field (H)=9.4 T, the temperature=22° C., the number of acquisition (NEX)=1, the field of view (FOV)=25×25 mm², the matrix size=128×128 voxels, echo train length=16, echo spacing=8.1 ms, slice thickness=2 mm, seven different repetition times (TRs) were used in linear increments from 200-2000 ms, and the echo time (TE)=32.42 ms. Signal was fit to the following equation using MATLAB to find T1:

$$S = {S}_{0} ( 1 - {e}^{- \frac{TE}{T 1}} )$$

where, S is the signal for a given voxel, and S₀ is the signal of that voxel at saturation. T2 mapping was carried out using a multi-echo scan with the same parameters as the T1 scan with the following exceptions: the number of echoes=10, 10 TEs linearly spaced from 10-100 ms, and TR=3000 ms. T2 mapping was performed using Osirix and linear fitting followed in MATLAB.

### Example 6—Animals

Both male and female young (6-8 weeks) C57BL/6J mice (Jackson Laboratory, Bar Harbor, ME) and middle aged (12 months) C57BL/6J and CX3CR1-GFP (Jackson Laboratory, Bar Harbor, ME) from a C57BL/6J background mice were used. Mice were housed in a 12 h/12 h day/night cycle with ad libitum access to standard mouse chow and water. All animal work was approved by and performed under the guidance of the University of Nebraska-Lincoln IACUC.

### Example 7—K^{trans} MRI

In vivo NP assessment consisted of dynamic contrast-enhanced (DCE)-MRI using a 9.4T MRI system (Varian) to compare targeting agent effect on the uptake as previously described (Miller et al., 2019, Scientific Reports, 9:16099). Briefly, mice were induced with about 2% isoflurane gas and maintained at a concentration sufficient to achieve 50 to 80 breaths per minute. Baseline T1 maps were generated using a gradient-echo sequence and the variable flip angle method with two angles, 10° and 30° (Deoni et al., 2003, Magn. Reson. Med., 49:515-26). Mice were injected with 100 μL of a 0.5 mM NP solution via tail vein catheter followed by 100 μL PBS to flush all remaining NP solution. A flip angle of 30° was used for all post-contrast scans, which occurred for 1 h following injection. TR was between 54 and 84 ms, TE between 2.73 and 4.24 ms, matrix size between 128×128 and 256×256, FOV between 20×20×10 mm³ and 25×25×10 mm³ depending on the experiment. NEX varied with matrix size to maintain temporal resolution, with 128×128, NEX=5 for all scans, with 256×256, NEX=4 for baseline and 1 for post-contrast scans.

Following serial image acquisition, resulting in about 100 post-contrast scans per animal, R1, concentration, and K^{trans} mapping were performed using custom MATLAB script. R1 mapping was performed using the variable flip angle method based on the following equation (Deoni et al., 2005, Magn. Reson. Med., 53:237-41:

$$\frac{{S}_{SPGR}}{sin ( α )} = \frac{{S}_{SPGR}}{tan ( α )} E 1 + {M}_{1} ( 1 - E 1 )$$

where S_{SPGR} is signal intensity, α is FA, E1 is exp(TR/T1), and M₀ is a proportionality factor related to longitudinal magnetization. E1 describes the linear relationship between the two signal intensity ratios, taking the slope, m, of that line enables calculation of T1 as:

$$T 1 = \frac{- TR}{ln ( m )}$$

Concentration maps were then generated by comparison of baseline R1 maps with post-contrast R1 maps using the following equation:

$$C ( t ) = \frac{R 1 ( t ) - R ( {t}_{0} )}{r 1}$$

where C(t) is the concentration at time t, R1(t) is the post contrast R1-value at t, RT(t₀) is baseline R1, and r1 is relaxivity of the contrast agent. K^{trans}, the contrast extravasation rate constant, mapping was then performed using a reference region (RR) model derived from the extended Tofts' model (Tofts et al., 1999, J. Magn. Reson. Imaging, 10:223-232):

$${C}_{TOL} ( T ) = \frac{{K}^{trans , TOL}}{{K}_{trans , RR}} * {C}_{RR} ( T ) + \frac{{K}^{trans , TOL}}{{K}_{trans , RR}} [ ( \frac{{K}^{trans , RS}}{{v}_{0} RR} ) - ( \frac{{K}_{trans , ROI}}{{v}_{eTOL}} ) ] * {\int }_{0}^{T} {C}_{RR} ( t ) {e}^{( \frac{- {K}^{trans , ROI}}{{v}_{0} TOL} ) ( t - t )} dt$$

where C_{TOI} and C_{RR} are concentrations in the tissue of interest (TOI) and the RR, respectively, K^{trans,TOI} and K^{trans, RR} are K^{trans} in the TOI and RR, respectively, and v_{e,TOI} and v_{e,RR} are extravascular-extracellular volume fractions for the TOI and RR, respectively. Muscle tissue from within the FOV was used in all animals as the RR. MATLAB was used to execute a least squares curve fitting routine to calculate K^{trans} for each voxel in the brain.

### Example 8—Microscale Thermophoresis (MST)

Human claudin-1 was expressed and purified as reported previously (Vecchio & Stroud, 2019, PNAS USA, 116:17817-24). MST measurements were performed using a Monolith instrument (NanoTemper Technologies). For experiments with human claudin-1, the assay buffer contained 10 mM Tris HCl, pH 8.0, 100 mM NaCl, 4% glycerol, and 0.04% n-Dodecyl-β-D-Maltopyranoside (β-DDM). NPs and peptides were labeled with AF647 prior to MST analysis as described above. For the binding experiments, the concentration of human claudin-1 was varied from 2.2-73,500 nM and the AF647-labeled C1C2-NP was fixed at 70 nM, while the AF647 labeled NPs and AF647 labeled C1C2 peptide were fixed at 1 and 0.3 μM, respectively. Binding of the AF647 labeled C1C2-NP to human claudin-3, -4, and -9 were tested using 0.4-14000 nM, 0.8-28000 nM, and 0.6-20000 nM, respectively; with the AF647 labeled C1C2-NP fixed at 70 nM. This assay buffer was similar, except β-DDM was replaced with 0.1% n-Undecyl-β-D-Maltopyranoside (beta-UDM). In order to check non-specific binding of β-DDM micelles to the AF647 labeled C1C2-NP, the concentration of beta-DDM was varied from 0.03-900 μM and the concentration of the AF647-labeled C1C2-NP was fixed at 1 μM. A similar experiment was performed wherein the concentration of beta-UDM was varied from 0.3-10,000 μM and the concentration of AF647-labeled control NP was fixed at 1 μM. The non-specific binding of human claudins 4 and 9 to the NP was examined by varying the concentrations of human claudins 4 and 9 from 0.7-20,100 nM and 0.8-31,100 nM, respectively. The human claudin sub-dilutions were mixed with the NPs or peptides and loaded in standard monolith NT.115 capillary tubes for measurement. All experiments were conducted at 23° C. using the nano-red channel of the Monolith instrument and the data were analyzed by using either Monolith analysis software or Graph Pad Prism, version 9 (Graph Pad Software, San Diego, California).

### Example 9—In-Vitro Analysis Over C1C2-NP Binding Towards Claudin-1

bEnd.3 cells were cultured according to the ATCC culture conditions in DMEM. These cells were subjected to ethanol (50 mM) treatment to induce claudin-1 expression and incubated with C1C2-NP for 4 hrs. For determining attachment of the C1C2-NP to human claudin-1, human claudin-1 encoded PCMV script plasmid was overexpressed in bEnd.3 cells and then incubated with C1C2-NP for 4 hrs.

### Example 10—Fluorescence Imaging

A fluorescence microscope (LSM800, Zeiss) was used to take the fluorescence image of mice brain. Mice were transcardially perfused after 2 h of MRI with 4% paraformaldehyde (PFA) in Dulbecco's phosphate-buffered saline (DPBS, Thermo Fisher Scientific, Waltham, MA). Brain tissue was collected, trimmed, and fixed in 4% buffered PFA for 24 h. The brains were moved into 30% sucrose in DPBS for 3 d at 4° C. for cryoprotection. The brains were then embedded in 2.6% carboxymethylcellulose (CMC, C4888, Sigma-Aldrich, St. Louis, MO), frozen on dry ice, and sliced coronally at a 15 μm thickness with cryotome (Leica Biosystems, Wetzlar, Germany). The brain slices were laid on poly-L-lysine coated microscope slides (Thermo Fisher Scientific) and dried overnight at RT. Sections were washed with DPBS thrice for 5 min each to remove the CMC. The brain slices were blocked with 3% normal donkey serum, 0.3% triton X-100, and 0.1% sodium azide in DPBS for 1 h at RT. The primary and secondary antibody (Ab) were diluted in the blocking buffer. The brain sections were incubated with 1:100 dilution of rabbit primary Ab against claudin-1 (51-9000, Invitrogen, Carlsbad, CA) for 2 d at 4° C., then washed thrice for 5 min each with blocking buffer before the sections were incubated with a 1:250 dilution of an AF555 labeled donkey anti-rabbit secondary Ab (ab150074, Abcam, Cambridge, UK) for 2 h at RT. The brain sections were again washed thrice for 5 min each with the blocking buffer before being stained with DAPI for 5 min, washed with DPBS followed by water, and mounted with ProLong™ Gold Antifade Mountant (Thermo Fisher Scientific).

Images were acquired with confocal microscopy at 40× objective lens magnification. Quantitative image analysis was performed with ImageJ software on at least two randomly selected viewing fields for each region for each mouse. The fluorescence signal of claudin-1 was thresholded to only integrate the density above 20 to remove the background fluorescence. Gamma correction was performed only on the representative images after the immunofluorescence quantification with shadows value of 0, midtones value of 0.26, and highlights value of 100 for all CMYK channels with Adobe Photoshop.

### Example 11—Statistical Analysis

All the data in this study were expressed as mean±standard error of the mean (SEM). A p<0.05 was considered statistically significant. The correlation between the integrated density of claudin-1 immunofluorescence and NPs K^{trans} value was evaluated using linear regression and one-tailed Pearson correlation coefficient with GraphPad Prism 7 software (GraphPad Software, CA). Data fitting for MST measurements were performed with GraphPad Prism 9.

### Example 12—Nanoparticle Synthesis and Characterization

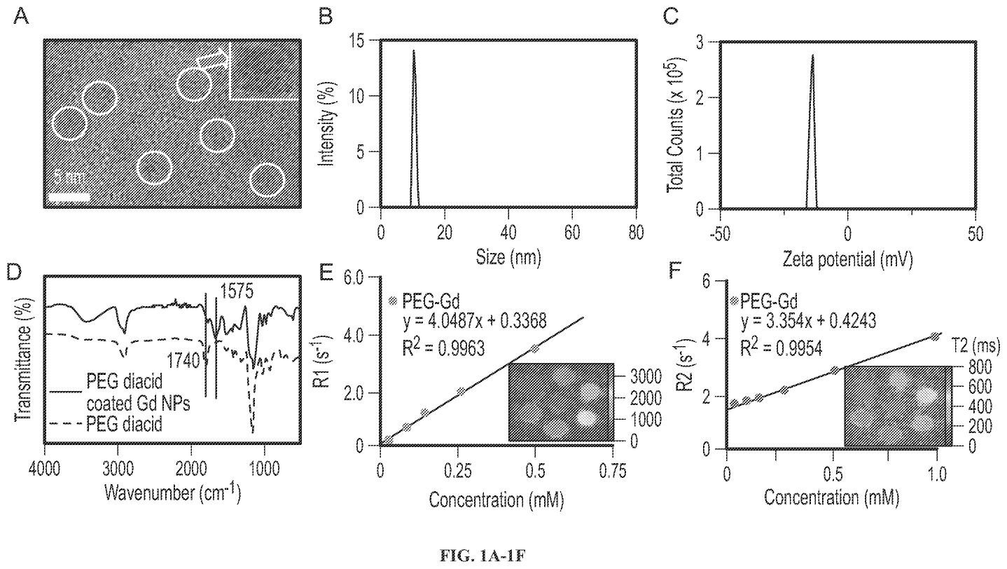

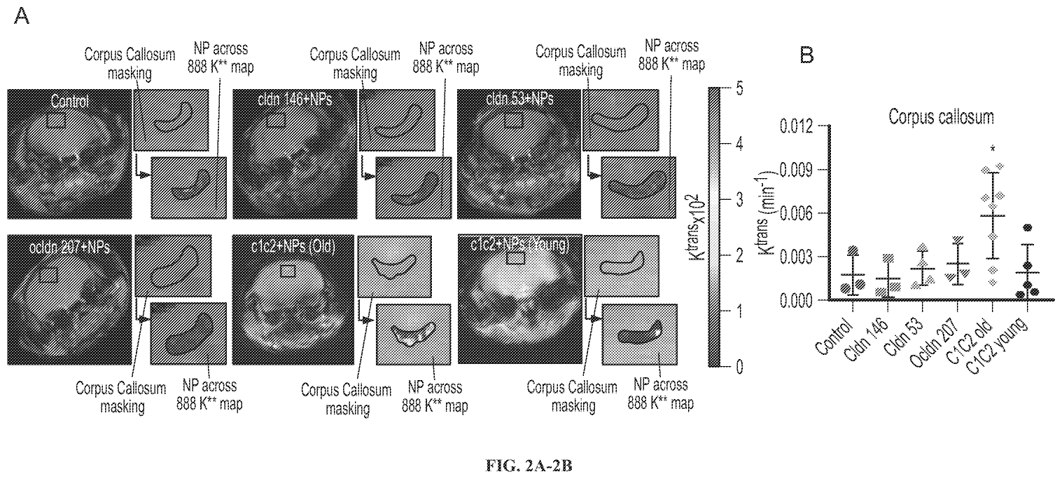

PEG diacid was used to coat the synthesized NPs. The addition of PEG to NPs surfaces can reduce clearance by the reticuloendothelial system (RES) and increase circulation time. Not only can the PEG coating prevent aggregation, but it can also increase solubility in serum because of repeating the hydrophilic ethylene glycol units. The high-resolution TEM images of PEG diacid coated Gd NP cores revealed the average NP diameter was around 3.5 nm, verifying their ultrasmall size (FIG. 1A). From DLS measurements, the average hydrodynamic size of the nanoparticle was 10.5 nm (FIG. 1B), and the zeta potential was −16.1 mV (FIG. 1C). The PEG diacid coating was confirmed using FTIR. PEG diacid-coated Gd NPs were compared with free PEG diacid and it was observed that the C═O stretch was red-shifted by ˜165 cm⁻¹ from that of a free PEG diacid (=1740 cm⁻¹) (FIG. 1D), confirming the attachment of —COOH group to the NPs as commonly observed in metal oxide NPs coated with —COOH group containing ligands.

To determine the MRI enhancing properties of the NPs, both R1 and R2 were measured at 9.4 T and plotted as a function of Gd concentration. Longitudinal (r1) water proton relaxivities were estimated from the corresponding slopes, giving a value of 4.05 s⁻¹mM⁻¹, and transverse (r2) water proton relaxivities were estimated from the corresponding slopes, giving a value of 3.35 s⁻¹ mM⁻¹ for PEG-Gd NPs. T1 and T2 map images (FIGS. 1E and 1F) show apparent dose-dependent contrast enhancement.

Peptides were conjugated to PEG on the surface of the NPs through EDC-NHS chemistry. Peptide density on the surface of the NP was between 15-19 peptides per NP (Table 1) depending on the peptides, indicating similar peptide densities of around 2.02, 2.14, 2.56 and 2.02 nm²/peptide, respectively. The hydrodynamic sizes and zeta potentials of all peptide conjugated NPs were similar (Table 1), reducing the chances of confounding cell binding and uptake effects caused by different physicochemical properties of the NPs.

TABLE 1
Properties of NPs and peptides

| | | | | Zeta |
|---|---|---|---|---|
| | | | No. of | Potential, |
| | | Hydrodynamic | peptides | mV |
| Name | Sequence of peptides | size (nm) | per NP | (with NPs) |
| Cldn146- | QEFYDPLTPINARYE | 13.94 ± 0.08 | 19 | 1.62 ± 0.13 |
| NP | (SEQ ID NO: 3) | | | |
| Cldn53-NP | SCVSQSTGQIQCKVFDSLLNLNSTLQAT | 13.8 ± 0.11 | 18 | 2.24 ± 0.18 |
| | (SEQ ID NO: 4) | | | |
| Ocldn207- | GSQIYMICNQFYTPGGTG | 14.23 ± 0.11 | 15 | 3.65 ± 0.19 |
| NP | (SEQ ID NO: 5) | | | |
| C1C2-NP | SSVSQSTGQIQSKVDSLLNLNSTQATR | 14.5 ± 0.13 | 19 | 7.81 ± 0.38 |
| | (SEQ ID NO: 6) | | | |

### Example 13—MRI Assessment of Brain Accumulation and Retention

DCE-MRI represents a robust method for assessing NP accumulation and retention in the brain as compared to more static methods such as concentration mapping or fluorescence imaging at single time points, which cannot distinguish between NPs in the blood or NPs that have been transferred to the tissue compartment. While DCE-MRI has typically been used to measure permeability of tissues, a method has recently been reported for comparing the permeabilities of NPs within a tissue using MRI to identify the permeability coefficient K^{trans}. The effect of peptide targeting on NP accumulation and retention in the brains of 1-year-old mice was assessed in brain regions known to be affected by BBB breakdown during aging (i.e., the corpus callosum and hippocampus, hypothalamus) and in regions thought to be minimally affected by aging (i.e., muscle, cortex). In the corpus callosum (FIG. 2), NPs targeted with the C1C2 peptide against claudin-1 (C1C2-NPs) showed a significant increase in K^{trans} in old mice as compared to other peptide modified and control NPs as well as compared to young mice, suggesting preferential binding to upregulated surface claudin-1 induced by aging. Similarly, in the hippocampus (FIG. 3), C1C2-NPs had a trending increase in K^{trans} that was not significant because of high variability of K^{trans} values in these aged mice that could be a result of differential claudin-1 expression between mice. A summary of K^{trans} results for each brain region is shown in Table 2. In the corpus callosum of aged mice, the mean K^{trans} value for control NPs was 0.0017 min⁻¹, similar to that observed in 12-16 month old mice using Magnevist as the contrast agent. The mean K^{trans} value for C1C2-NPs was significantly greater at 0.0058 min⁻¹, suggesting active binding to increased claudin-1 expression. In the hippocampus of aged mice, the mean K^{trans} value for control NPs was 0.0018 min⁻¹, similar to a K^{trans} of ˜0.001 min⁻¹ observed for middle aged humans of around 40 years of age using MultiHance as the contrast agent. For C1C2-NPs, a K^{trans} value of 0.0040 min⁻¹ was observed in the hippocampus. The highest K^{trans} value in the cortex and hypothalamus of young or/and aged mice was 0.0145 min⁻¹ and 0.0134 min⁻¹ for C1C2-NPs. On the other hand, the highest K^{trans} value in the muscle was observed for control mice as 0.0079 min⁻¹.

TABLE 2
Brain region specific accumulation and retention of NPs targeted with various peptides
as measured using the permeability coefficient, K^{trans} (min⁻¹), with MRI. CC,
corpus callosum; HC, hippocampus; CTX, cortex; HT, hypothalamus; muscle

| | | NP Type | | | | | |
|---|---|---|---|---|---|---|---|
| | | | | | | C1C2- | C1C2- |
| | | | Cldn146- | Cldn53- | Ocldn207- | NP | NP |
| | | Control | NP | NP | NP | (Old) | (Young) |
| Brain | CC | 0.0017 ± | 0.0014 ± | 0.0022 ± | 0.0025 ± | 0.0058 ± | 0.0019 ± |
| Region | | 0.0014 | 0.0012 | 0.0012 | 0.0014 | 0.0030 | 0.0019 |
| | HC | 0.0018 ± | 0.0012 ± | 0.0018 ± | 0.0019 ± | 0.0040 ± | 0.0019 ± |
| | | 0.0012 | 0.0005 | 0.0085 | 0.0014 | 0.0030 | 0.0017 |
| | CTX | 0.0016 ± | 0.0003 ± | 0.0026 ± | 0.0020 ± | 0.0148 ± | 0.0022 ± |
| | | 0.0010 | 0.0003 | 0.0009 | 0.0021 | 0.0067 | 0.0015 |
| | HT | 0.0012 ± | 0.0008 ± | 0.0026 ± | 0.0037 ± | 0.0102 ± | 0.0026 ± |
| | | 0.0015 | 0.0002 | 0.0013 | 0.0013 | 0.0056 | 0.0017 |
| | Muscle | 0.0079 ± | 0.0016 ± | 0.0009 ± | 0.0033 ± | 0.0057 ± | 0.0028 ± |
| | | 0.0022 | 0.0013 | 0.0013 | 0.0007 | 0.0022 | 0.0029 |

### Example 14—NP Accumulation and Retention Correlates with Claudin-1 Expression

Higher K^{trans} value of NPs were found in the old mice than in the young mice, but there was high variability between animals, so immunofluorescence staining of claudin-1 was performed to observe the expression of claudin-1 in the brains of old and young (FIG. 4). A significant correlation was found between K^{trans} and claudin-1 expression in the mice brain in the corpus callosum, CAT, CA3, and dentate gyrus (r=0.6622, 0.8826, 0.9039, and 0.8487 with p=0.037, 0.002, 0.001, and 0.004, respectively). These result suggested that NPs accumulation correlates with the claudin-1 expression in the mice brain, which might be caused by the NPs active targeting toward claudin-1 on the BBB. Therefore, this provides strong evidence of variability in claudin-1 expression in these aged mice, which was observed in the K^{trans} imaging with the C1C2-NPs.

### Example 15—Specific Binding and Affinity of C1C2-NP to Claudin-1

To determine whether C1C2-NPs specifically bind claudin-1, mouse brain microvascular endothelial (bEnd.3) cells were exposed to the AF-647-modified NPs in vitro. Control cells showed no binding to C1C2-NPs as expected, since claudin-1 expression is low under normal culture (FIG. 5A-C). When claudin-1 expression was induced by exposure to ethanol, C1C2-NPs showed much higher binding to cells, suggesting specific binding to mouse claudin-1. To determine if C1C2-NPs could also bind to human claudin-1, bEnd.3 cells were transfected to express human claudin-1 and exposed to C1C2-NPs. High NP binding was observed in this condition, suggesting C1C2-NPs can bind to both human and mouse claudin-1.

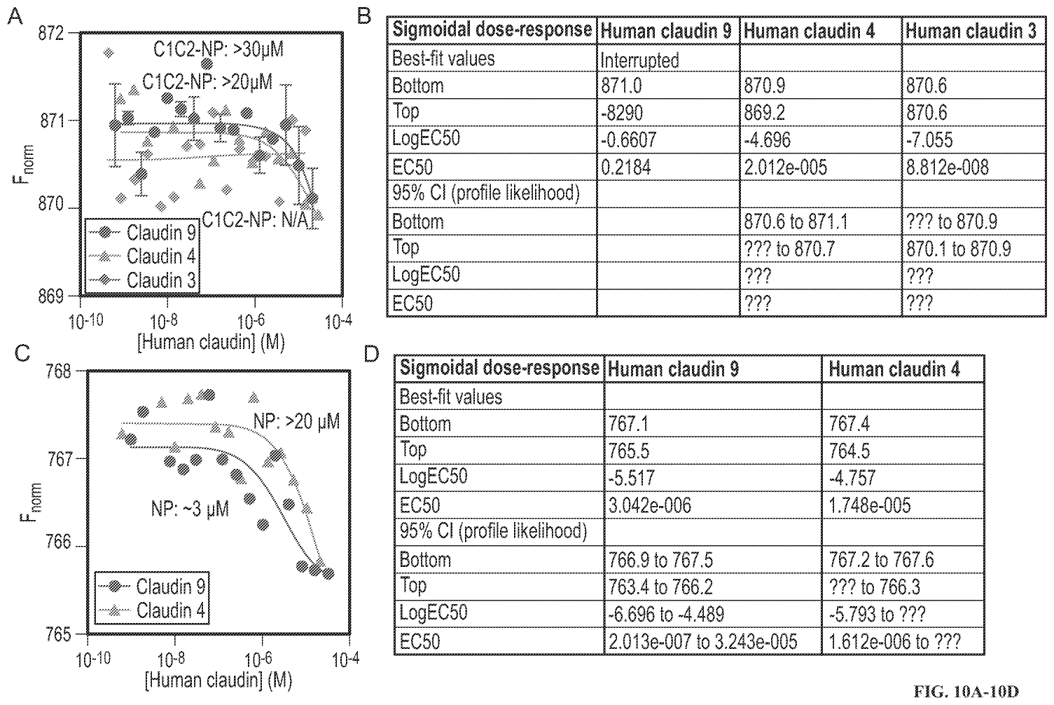

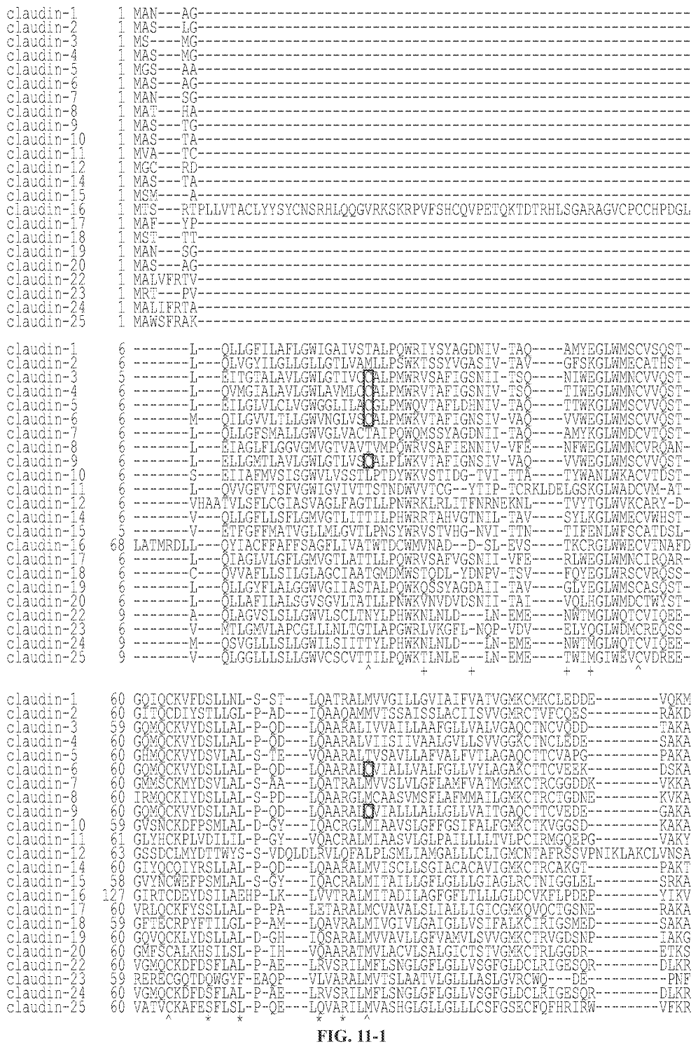

To assess the binding affinity of C1C2-NPs to human claudin-1, C1C2-NPs were labeled with AF647 and binding to recombinant human claudin-1 quantified using MST. The dissociation constant (K_{D}) of C1C2-NPs to human claudin-1 was 21±14 μM (FIG. 5D). Measuring the binding of empty NPs to human claudin-1 found a K_{D} of 335 μM, while no binding to human claudin-1 was detected for C1C2 peptides alone. In order to rule out non-specific binding of the AF-C1C2-NP to the j-DDM micelle, the concentration of 3-DDM was varied and the concentration of AF-C1C2-NP was fixed at 1 μM. No significant binding could be observed within the range of 3-DDM concentration used for the measurement with human claudin 1 (data not shown). The specificity of the binding of AF647-C1C2-NP was verified by measuring its binding to the human claudins 3, 4 and 9. Human claudin 3 does not show any binding. The data points for human claudins 4 and 9 could not be fit to the sigmoidal dose response curve suggesting that the binding of AF647-C1C2-NP to human claudins 4 and 9 is weak (FIG. 10A, 10B). However, both human claudins 4 and 9 bind to AF647-NP (FIG. 10C, 10D) and this does not represent non-specific binding of the nanoparticles to the detergent micelles. These data indicate that the presence of C1C2 peptide makes the nanoparticle-peptide conjugate specific for binding to human claudin 1 as well as mouse claudin 1 from the in vitro experiments.

### Example 16—NPs were Observed in Regions of High Claudin-1 Expression

Since there is a significant correlation between NPs accumulation and claudin-1 expression, the localization of the NPs in the brain were assessed with confocal microscopy and it was found that NPs co-localize with the immunofluorescence staining of claudin-1 (FIG. 6). This strongly supports the idea that the C1C2-NPs are able to bind to regions of increased claudin-1 on the BBB.

The experimental methods described herein with claudin-1 can be used with any of the other claudin sequences or with occludin.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

1. A nanoparticle comprising a claudin or occludin polypeptide or portion thereof conjugated thereto, wherein the nanoparticle carries a payload.

2. The nanoparticle of claim 1, wherein the claudin polypeptide is selected from claudin-1, claudin-3, claudin-5, claudin-11, claudin-12, claudin-25, and claudin-27.

3. The nanoparticle of claim 1, wherein the claudin polypeptide is claudin-1.

4. The nanoparticle of claim 3, wherein the claudin-1 polypeptide has the amino acid sequence shown in SEQ ID NO:2.

5. The nanoparticle of claim 4, wherein the portion of the claudin-1 polypeptide is selected from Cldn146 (SEQ ID NO:3) or Cldn53 (SEQ ID NO:4).

6. The nanoparticle of claim 1, wherein the payload is an imaging agent.

7. The nanoparticle of claim 6, wherein the imaging agent is Gd.

8. The nanoparticle of claim 6, wherein the imaging agent is a radiotracer.

9. The nanoparticle of claim 1, wherein the payload is a therapeutic agent.

10. The nanoparticle of claim 9, wherein the therapeutic agent is a bispecific antibody, a monoclonal antibody, an immunoglobulin fragment, an immunotoxin, a nucleic acid, a protein (e.g., a recombinant protein), a drug, a small molecule, a pharmaceutical, or combinations thereof.

11. The nanoparticle of claim 1, wherein the nanoparticle is an ultrasmall nanoparticle.

12. The nanoparticle of claim 1, wherein the nanoparticle is about 3.5 nm in diameter.

13. The nanoparticle of claim 1, wherein the NPs are coated with PEG diacid.

14. A method of delivering a therapeutic to or across the blood brain barrier (BBB) in an individual, comprising:
delivering the nanoparticle of claim 1 to the individual, wherein the payload is a therapeutic.

15. A method of imaging the blood brain barrier or determining the permeability of the blood brain barrier in an individual, comprising:
administering the nanoparticle of claim 1 to the individual, wherein the payload is an imaging agent.

16. The method of claim 14, wherein the individual is experiencing neurocognitive decline.

17. The method of claim 14, wherein the individual is at risk of experiencing neurocognitive decline.

18. The method of claim 14, wherein the individual has or has had a brain tumor.

19. The method of claim 14, wherein the individual has or has had a traumatic brain injury (TBI).

20. The method of claim 14, wherein the individual has been diagnosed with a neurological disorder.

21. The method of claim 20, wherein the neurological disorder is Alzheimer's disease.

22. The method of claim 14, wherein the administering step is via oral administration.
